(19) United States
(12) Patent Application Publication
(10) Pub. No.: US 2022/0317117 A1
(43) Pub. Date: Oct. 6, 2022

(54) System and Method for Dual Bio-Sensor Fabrication and Use

(71) Applicant: Richard Postrel, Miami Beach, FL (US)

(72) Inventor: Richard Postrel, Miami Beach, FL (US)

(21) Appl. No.: 17/705,311

(22) Filed: Mar. 26, 2022

Related U.S. Application Data
(60) Provisional application No. 63/167,465, filed on Mar. 29, 2021.

(51) Int. Cl. G01N 33/543; B01L 3/00; B01L 7/00

(52) U.S. Cl. CPC G01N 33/5438; B01L 3/502761; B01L 3/502715; B01L 7/00; G01N 27/02

(57) ABSTRACT

The present invention provides a system and method for building and optimizing biosensors to create a multi-layered bio-sensing system by incorporating two different formats comprised of i) a single wall carbon tubular sensing element and ii) a non-tubular graphene sensing element. This multi-layered system allows for assaying molecules across a large range of molecular weights by sensing molecules in both gas and liquid from a common sample simultaneously. By collecting and analyzing both larger, heavier molecules, including, but not limited to: proteins hormones, nucleic acids, lipids, lipoproteins, etc., with non-tubular graphene sensors and smaller, lighter volatile organic compounds (VOCs) as emitted in gas form from the same sample are assayed with single walled-carbon nanotubules (SWNTs), this invention provides a more complete, holistic understanding of the organism's current state of health.

The present invention provides a system and method for building and optimizing biosensors to create a multi-layered bio-sensing system by incorporating two different formats comprised of i) a single wall carbon tubular sensing element and ii) a non-tubular graphene (ntG) sensing element. This multi-layered system allows for assaying molecules across a large range of molecular weights by sensing molecules in both gas and liquid from a common sample simultaneously. By collecting and analyzing both larger, heavier molecules, including, but not limited to: proteins hormones, nucleic acids, lipids, lipoproteins, etc., with ntG sensors and smaller, lighter volatile organic compounds (VOCs) as emitted in gas form from the same sample with single walled-carbon nanotubules (SWNTs), this invention provides a more complete, holistic understanding of the organism's current state of health.

The modular arrangements of the present invention provide multiple sensing surfaces with specific areas or zones for detection of organic metabolites and evaporations from a common sample. The ntG sensors provide sensing capacities for larger, heavier, and non- or less-volatile compounds (nVOCs) while the carbon nanotubular sensors (SWNTs) provide sensing capacities for the smaller lower molecular weight (MW) compounds.

Smaller, lighter molecules generally have less interactive surface area and thus higher partial pressures favoring a gas state that correlates with lower MWs (generally less than 400 Da) while larger heavier molecules have greater interactive surface area with MWs (generally greater than 400 Da). Smaller compounds favor a gas phase assay while larger compounds favor the liquid phase. Partitioning is not absolute. Molecules of the same compound often will be assayed in both liquid and gas.

The VOCs and nVOCs of interest are metabolites resulting from an organism's life sustaining biochemical reactions, including anabolic, catabolic, athletic, immunologic, etc., functions. that are dissolved in the body's aqueous matrix, with VOCs being emitted (or off-gassed) from the biological source material. For sampling, body fluids as well as biological source materials (solid biopsies) themselves, are delivered into a system where a module or chip responds electronically when individual molecules approach and remain in close proximity to surfaces of one or more sensing elements. Sensing elements may be modified (or decorated) with molecules that may attract, repel, or have no interaction with a passing VOC or a larger molecule which may have been delivered in a gas or liquid.

The preferred embodiments comprise at least 2 separate sensing platforms, in a parallel or stacked 3-D arrangement. By incorporating both of these embodiments in a singular device, the invention optimizes capture, classification, and pattern recognition necessary for identifying molecular signatures unique to each disease, condition, or factor of interest and may further provide valuable information with respect to preferred treatment therapies. Various and differentiated sensor elements provide multiple opportunities for each passing VOC or non-volatile compound to interact with a sensor. The manner of electronic differential (sensed strength of interaction, time of interaction, etc.) with each sensor element or portion thereof helps to further distinguish various interactions and thereby to more robustly differentiate the collected interactive events.

### BACKGROUND

Different compounds are characterized by length and/or strength of interactions with each of the sensors or sensor types. A stronger affinity for a specifically decorated sensor will slow movement and increase time a molecule is sensed. Summing all these interactive events from a sample together builds a picture or signature of the multitude of individual compounds that pass through the device. The patterns of totality of interactions of the various differently interacting compounds provide signatures that characterize different diseases, disease states, and health status of individuals.

The present invention features a novel formation of sensors constructed as stacked layers that may appear as lower planar graphene layers, sensor layers of Single Walled-carbon NanoTubes (SWNTs), layers with cone or pyramidal shape sensors rising from a base of the sensing plane. Graphene is an allotrope of carbon where carbon atoms form a single sheet of a two-dimensional honeycomb lattice nanostructure. Each atom in the graphene sheet is bonded to three nearest neighbors by a σ-bond. Graphene can be made as a two dimensional sheet with sides (edge carbons) either triple bonded or with an unpaired electron. The SWNT allotrope is a graphene form where edge carbons are eliminated through the sheet curving to close the edges into a tubular structure. Thus, graphene allotropes can be planar (with edge carbons on all sides) or tubular (with edge carbons at the tubular ends). Tubular forms may be in a single layer (SWNT) or in a multi layer, usually concentric, configuration.

The primary system of the invention features a flat (non-tubular) graphene sheet as a first sensor layer with a second sensor layer comprising tubular graphene coiled as SWNTs. The second sensor layer may be essentially two dimensional (2-D) or may be formed as a stacked pyramidal structure. The pyramidal layers can be deposited one atop another or formed in a continuous spiraling roll. A multi-layer nanosensing device of the present invention takes on one of three basic formats. A) a planar graphene layer for assaying molecules (compounds) present in a liquid sample and a single layer of SWNTs that assay VOCs in their gas phase. B) a pyramidal disposition of SWNTs with stacked multi-layers providing a 3-D formation multiplying sensing surface over the sensor base providing strong sensitivity and differentiation capabilities for assaying VOCs in their gas phase, and C) a planar graphene layer for assaying molecules (compounds) present in a liquid sample and the pyramidal disposition of SWNTs of B).

The systems involve application of planar-bonded carbon structures such as graphene and single wall nanotubules (SWNTs). The “flat” carbon component atoms of the graphene or rolled graphene as nano-tubules are receptive to complexing with cyclic, preferably heterocyclic, chemical structures (decorations or functionalizations), often occurring through a non-covalent π-bonding effect. Graphene, planar or crumpled, and SWNTs having similar single layer carbon geometry, have selectivity differentiated by binding to select, e.g., different species of nucleic acid, decorations. Other materials including, but not limited to: transition metal dichalcogenides, hexagonal boron nitride, black phosphorus, metal oxides, synthetic polymers, etc., may be constructed as signal control devices similar in use for functions with SWNTs and graphene and thus may be advantageously employed in certain circumstances. Overall, current evidence indicates that the curved carbon structures of the SWNTs demonstrate consistent with FET properties in many use environments and are especially sensitized with various functionalization (decoration) compounds. Therefore, curved graphene, possibly formed into a corrugated or spiral geometry, (See, e.g., Michael Taeyoung Hwang, et al., Ultrasensitive detection of nucleic acids using deformed graphene channel field effect biosensors. Nat Commun 11, 1543 (2020). doi.org/10.1038/s41467-020-15330-9) should demonstrate more promising specificity, speed of analysis, and/or sensitivity over planar graphene for particular applications. As nanotechnology continues to progress, additional sensor formats such as those emitting light, will become accepted in the art. Embodiments of the present invention may incorporate these improved sensors as their reliability is established. The skilled artisan will generally choose which form of sensor is optimal for performance and cost.

In addition to the field effect electrical sensing set forth as preferred embodiments, other qualities of thin carbon based used for sensing are possible. Optical, electrochemical and electrical features have been employed with graphene-based biosensors. Forms of graphene have been successfully tested for electrochemical (amperometric, voltammetric, impedimetric, or combinations thereof) and electrical sensing applications. Selected formats have the high electron transfer rate, the high charge-carrier mobility and manageable electrical noise that is necessary for sensitive detection of biomarkers and other biological analytes. Successful assays have been reported in both serum and blood extracts. Optical transparency of graphene monolayers allows use in sensors such as optical-based G-biosensors.

Embodiments of this invention may incorporate additional or alternative sensing element types. E.g., graphene in one of its more planar forms, curved, crumpled, etc., is incorporated as one of a plurality of sensor layers in a unified device that allows for larger and smaller molecules to be detected and characterized individually and simultaneously in the same device. The more planar ntG form is disposed to assay compounds in a liquid sample In the secondary, pyramidal systems, the decorated SWNTs, are adept as detectors for VOCs with a median value of about 400 MW and less, whereas in the primary system, a sensor based, e.g., on curved or crumpled graphene, can be constructed to preferentially interact with larger or longer molecules that are heavier and therefore generally less volatile. An interface,such as graphene, including essentially planar curved and crumpled forms can provide a greater opportunity for larger molecules to interact. Larger molecules tend to have more bonds that contribute to flexibility. These larger molecules may bend and conform to more spread out sensing elements for better characterization and detection. One format may include a graphene base layer with a pyramidal SWNT layer opposed as a parallel plane. For example, a liquid sensing layer may occupy the base and sense molecules in the liquid, while gases emitted from the liquid are sensed using the pyramidal sensor layer. A large molecule sensing layer may also be incorporated to detect larger molecules in a gas phase. The multi-detection formats may be in parallel or in series dependent on design preference. Serial formats include tubular designs, e.g., one where a larger molecule sensing portion is disposed close to an entry port. Optionally an inert zone intervenes between the large and small molecule favoring portions. Such zone may allow for controlling temperature. Such zone may incorporate non-inert surfaces that may be present to bind, i.e. filter or cleanse the sample of molecules that may reduce effectiveness of the downstream VOC detection elements.

The pyramidal sensors are preferably constructed as stacked or repeating layers of decorated carbons with each rising layer providing a small shelf and sensing ridge. In this formation each ascending layer has a smaller diameter that the one below. The term “diameter” is used loosely to represent a cross sectional surface area. For understanding the term diameter as it often appears in the application, the expression: A^{½}·π^{-½}·2 can be substituted for “diameter”, where A is footprint area. The general understanding is simply, the outer bounds of a layer more distal from the base are tighter than the outer bounds of the layer more proximal to the base. Thus a pyramid may take on a circular shape. However, footprint or shape is not a critical feature for sensing. Thus, an ellipsoid, a regular or irregular geometric shape, including, but not limited to: hexagons, triangles, rectangles, rhombuses, octagons, etc., may be a design choice. In the conical shape, each sensor is more proximal to neighbor sensor bases than their elevated portions. The distance between sensors increases with their heights. Gas flow is more impeded at the base due to both the sensor density and the flow inhibition provided by the immobile base. Where the base or lowermost layer of the sensing element is charged to produce an electric field field strength declines with height and thus provides an additional variable for consideration in forming the disease signature.

Where an electric field is produced by a charged spike at the center of the sensor element, the electric field is strongest at the top where the exposed ridge is closest to the center. The timing and strength of interactions thus varies with height. In embodiments where sensor elements are densely packed, competition for interaction between adjacent sensors decreases with height off the base surface. The interactions of different compounds with different sensors and at different heights are part of the distinguishing features defining the signatures. Where the sensor elements are constructed as tubes or spirals, flow through the sensor element arrays will still be affected by immobile walls and/or floors, bur the through flows at different heights off the base are variable with sensor diameter and separation distances.

Where the sensors may include, e.g., base portions engineered to respond to, i.e., interact with larger molecules, including liquids in some embodiments, the upper layers may detect emissions from the liquid source. Several of the detected emissions will correspond to compounds that may be dissolved or complexed in the liquid and may include homo or hetero dimers or polymers. In this formation both VOCs and non-VOCs including larger molecules are detected and analyzed simultaneously in the same sample run.

Historical Introduction

Diagnosis of many diseases can be expedited with availability of easily obtainable biosamples analyzed in a rapid turnaround assay device. One category of analyses concentrates on biomolecules produced by a living organism and/or a pathogen attacking the organism. In complex animals with immune systems, diagnosis is aided by conventional assays, such as blood tests, that might provide results including, but not limited to: counts relating to numbers and types of cells in the blood, sugar(s), electrolytes, proteins/peptides of interest—including peptides complexed with carbohydrates or lipids, specific DNA or RNA fragments, etc. The present invention expands this existing library of analyses through rapid assay of volatile organic compounds (VOCs) and at a higher level comparing the outputted signature information to a VOC signature library. In some embodiments, less volatile or non volatile compounds will be detected and characterized by sensor layers or elements that are engineered for more selective interactions with larger molecules, complexes or compounds.

Signatures can be analyzed independently, averaged, and/or in combination to suggest disease status and to thereby recognize a disease signature at the onset of the disease with maximum sensitivity and selectivity. By measuring signal amplitudes of the VOCs, and/or of larger compounds, the present device and methods can provide mathematical values for a multi-dimensional (multi-compound) interactions and gauge the status of a disease to allow, for example, a practitioner to assess the progress and efficacy of a particular treatment or treatments allowing for best practice to be significantly improved.

Signature comparisons will be useful in monitoring efficacy or progress of a chosen treatment, in identifying a pathogenic disease, monitoring a waxing or waning of a physical condition, like cancer, or those relating to emotions, aging, depression, cognition, etc. A compact device as provided in accordance with the present invention is capable of rapidly assaying easy to obtain non-invasive biosample material and outputting rapidly obtained analysis to determine relevance to disease of a particular patient's biosample. A library of VOC signatures for identifying various diseases and disease types as provided by this invention would constitute a useful addition to fields of medical and/or social well-being and serve as a warning against pathogenic disease and bioterrorism. The device of the present invention can also conduct VOC analysis to identify and measure exposure to harmful chemicals and to guide practices of avoidance or treatment, if necessary.

The present invention provides a device that assays multiple products produced by an organism's metabolic activities with a specificity sufficient to indicate presence of and highlight or identify abnormal, e.g., unhealthy chemical reactions. The abnormal metabolic reactions underlying production of the detected compounds may in some circumstances, be a general indication of declining or poor health or in others, may be more specific, i.e., indicative of a class of diseases or of a specific disease. A principle feature of this invention is an ability to rapidly obtain and analyze data from the assay of gaseous components that are contained in or that off-gas from a biologic sample and then to rapidly determine the presence of a specific disease.

With respect to VOCs, several common off-gases have historically been recognized as indicators of disease and the present invention builds upon this ancient knowledge. “Vapors” or odors have long been recognized as useful indicators of disease. For example, the ketosis exemplary of diabetes is recognized in early writings over three thousand years old. Egyptian manuscripts from around 1550 BC recognize diabetic ketosis and sweet urine. Sweet urine and ketone breath along with polyuria were recognized as a signal of an impending death. Unani physicians today rely chiefly on urine, especially its smells, to aid disease diagnosis.

Today we know the underlying chemical and biological bases for diabetes and many other diseases. Multiple diseases have been associated with one or often a collection of various organic compounds. In the 20^{th} and 21^{st} centuries, increased sensitivity of electronic chemical detectors, often called “electronic noses” has allowed the use of “vapors” or odors to confirm or correlate with diagnoses for diseases including, but not limited to: prostate cancer, breast cancers, renal disease, diabetes, etc.

For example, Tanzeela Khalid, et al., in “Urinary Volatile Organic Compounds for the Detection of Prostate Cancer” published an article whose aim was to investigate VOCs emanating from urine samples with a goal of determining whether they could be used to classify samples into those from prostate cancer and non-cancer groups. They used mass spectrometry (MS) coupled with gas chromatography (GC) to analyze the headspace over collected urine as a sample source and thereby determined that using four specific VOCs in the detection regimen improved the accuracy of prostate detection over that of PSA alone. Similarly, in another study, urine was analyzed for content of over 60 VOCs to correlate patterns associated with breast cancers.

Urine is an excellent source for obtaining biosamples. The renal system uses the kidneys to filters and concentrate compounds from the circulating blood as it passes through the kidneys. Thus, since information from the entire body is present in the blood, urine can be used to obtain health information for the body as a whole.

Breath can also be used, as well as plasma, saliva, sweat, semen, mucous, lymph, feces, and extracts or lysed cells such as blood cells or cells obtained in biopsy of any organ, including, but not limited to: skin, liver, lung, kidney, muscle, etc. In practice, the invention can assay VOCs from gas or off-gas from any source of interest, including biological sources including, but not limited to: ambient air, urine, blood, tears, sweat, plasma, flatulence, lymph, semen, vagina secretions, feces, wounds and/or festering wounds, breath, saliva, gas (VOC) emissions from a collection of animals or people, an individual, any internal or external surfaces (including the armpits, scalp, sinuses, ear canal, ear folds, feet, navel or umbilicus), etc.

If a gas, like breath, is sampled, the breath itself is considered as a sample off-gas. Breath, a product of the GI and pulmonary systems, has a different variety of VOCs than circulation as a whole, and often has higher levels of contaminating VOCs from the environment. Gastrointestinal sources also incorporate significant quantities of VOCs that are produced by the microbiomes resident in and residing on the organism. The throats, nasal passages, navels, armpits, toe gaps, etc., will have organismic emissions as well as emissions from the resident microbiomes.

Since many VOCs in e.g., breath are emitted from a changing microbiome that may be rapidly mutating dependent on diet, rather than from the actual cells of the host organism, reliance on microbiome sourced gases without other geographic or cultural information may render a high degree of specificity more difficult to achieve. Organismic sourced compounds will tend to apply more broadly across populations. Personal information such as diet, living conditions, occupational exposures, etc., may therefore be used to more accurately characterize diseases or conditions an individual presents with.

Advanced Analytics

To date laborious techniques such as MS/GC have been the preferred analytical tool for VOC analysis. Improvements in detection sensitivity from micro-detection to nano-detection using highly advanced sensors now enables a more robust use of nano-analysis of VOCs and other compounds and when combined with rapid data analysis and machine learning can: a) confirm a diagnosis, b) assist in selecting or ranking diagnoses and/or c) suggest one or more diagnoses even prior to outward symptoms becoming apparent. After assay, in some circumstances simply questioning a patient about a result may elucidate an overlooked symptom of disease.

The general approach of monitoring VOCs for detecting disease has been in development for several decades and now is soundly acknowledged in developing science and health medicine. In accord with the present invention a device to achieve these VOC assay goals has been designed to assay a variety of volatile organic compounds (VOCs) in a rapid and reproducible fashion. Under the present invention, multiple disease signatures can now be searched from the same sample simultaneously. The basic benefits of measuring VOCs for disease detection have been recognized in medicine for quite some time. In a 2014 Clinical Policy Bulletin, Aetna explained its policy regarding VOC analysis at that time as:
Aetna considers the analysis of volatile organic compounds experimental and investigational for the following indications (not an all-inclusive list) because the clinical effectiveness of this technique has not been established:
Detection of bacteriuria
Detection of cancer (e.g., breast cancer, colorectal cancer, lung cancer and cancer of the pleura, pancreatic cancer; not an all-inclusive list)
Diagnosis of amyotrophic lateral sclerosis
Diagnosis of autism spectrum disorders
Diagnosis of inflammatory bowel disease
Diagnosis of juvenile idiopathic arthritis
Diagnosis of non-alcoholic fatty liver disease
Differential diagnosis of breast diseases (e.g., breast cancer, cyclomastopathy, and mammary gland fibroma)
Prediction of development of childhood obesity
Use as markers for monitoring hemodialysis efficiency¹ ¹Clinical Policy Bulletin: Analysis of Volatile Organic Compounds. Number: 0717. Revised April 2014. Obtained from: http://qawww.aetna.com/cpb/medical/data/700_799/0717_draft.html

### SUMMARY OF THE INVENTION

Urine, exhaled breath, and blood are recognized as available sample sources. The present invention may use these and/or samples including, but not limited to: saliva, perspiration, fecal material, earwax, skin (or other organ biopsy) as a source for the assay.

The device of the present invention provides rapid highly sensitive detection of VOCs in a gas phase sample. Analytical data are then processed using the device's library of algorithms to detect a disease or to answer questions for which the sample was taken. Through machine learning and artificial intelligence (Al), the device is continually developing and improving its algorithms. The device, as described herein, which is capable of providing signature information from a variety of assays, including bioassays or assays of structures suspected of emitting possible harmful compounds into the ambient atmosphere, meets multiple identified needs and applications.

Through capturing the VOCs in a vapor or gas phase and optionally ,other compounds from a denser source, to measure the presence, amounts, volume, and/or intensity or strength of signal of multiple compounds; classifying each signal as from the organism or from the environment to remove especially the foreign VOCs from consideration, the device then produces a report characterizing the organism initiated organic compounds. This report is available for comparison to a signature database comprising signatures fro multiple diseases and conditions which thereby can determine whether a specific disease (or set of diseases) is present in that individual. Aspects of the present invention may continue to consolidate VOC, nVOC and other signature profiles into one or more libraries as each new sample output is presented.

In addition, the device may physically incorporate add-on devices and/or applications, for example, a capillary analytical attachment, including, but not limited to: capillary electrophoresis, capillary chromatography, capillary ELISA, nano-sensors similar to the vapor phase sensors but proximal to analyte in a liquid phase, etc. Add-on devices may be analytical providing additional information to be used in data analysis and signature identification or in some embodiments may absorb, adsorb, catalytically modify and/or filter out potential confounding compounds and thereby minimize the necessity for applying algorithms to remove the undesired ambient VOCs. The machine learning component of the invention, in preferred embodiments, has capacity for inclusion of externally generated information from add-on devices and/or from externally provided information.

One preferred format of the present invention features “chips” with modular nano-sensing elements (or nano-sensor element (NSE) that are independently maintained at a fixed, fluctuating, stochastic, alternating, discontinuous or flashing feeder power supply. The outputs of each NSE sensor element may be individually wired to a dedicated data transducer or a selection of sensor outputs may use a common carrier circuit and thus be “averaged”. In some embodiments, a simpler circuitry may involve multiple elements feeding a single output that may sum the outputs of multiple sensor elements to deliver an averaged reading. When one or more of the “averaged” sensors is turned off or powered down, the average will not include output from these one or more powered down sensors. When input sensors are powered individually, for example, in a cycling pattern when only one (or a selected portion) of the input electrodes is being charged, averaged outputs synchronized with the timing of input charging can thus provide data from individual channels. Nanosensing elements on a chip may be specialized for VOC characterization or for larger molecules, or a sensor element may incorporate portions engineered to sense both VOCs and less volatile molecules. Additional chips or blocks, rows, or columns on a chip may be modularly configured in desired formats to produce a multi-dimensional output from any given sample. Each sensor individually will produce its characteristic output as a contribution to the gross consolidated output.

The consolidated output channel in a specific task may connect and thereby collect data signal from any desired fraction of elements. For example, a single output may receive signal from all elements on a chip, half the elements on a chip, one-third the elements on a chip, a quarter the elements on a chip, a fifth the elements on a chip, and so on, for example, ⅙, 1/7, ⅛/, 1/9, 1/10, 1/12, 1/20, 1/25, 1/33, 1/50, 1/100, etc. An output may be associated with 1, 2, 3, 4, 5, 6, 7, 8, 9, 10, 11, 12, 13, 14, 15, 16, 17, 18, 19, 20, 21, 22, . . . , 24, . . . , 32, . . . , 48, . . . , 50, . . . , 64, . . . , 96, . . . , 100, . . . , 128, . . . , 200, . . . , 250, . . . , 256, . . . , 500, . . . , 512, . . . , 1000, . . . , 1024, . . . , 2048, . . . , 4096, . . . , 5000, . . . , 8192, . . . , 10,000 (10⁴), . . . , 16,384, . . . , 2¹⁵, . . . , 2¹⁶, . . . , 10⁵, . . . , 2¹⁷, . . . , 2¹⁸, . . . , 2¹⁹, 10⁶, . . . , 2²⁰, . . . , total number of sensors on a chip which may vary with time or programmed instructions. The precise count of sensor elements associated with any output in general is a design feature and does not define operative functions of the invention. The counts specifically exemplified above are exemplary low numbers of sensors that may feed an output and higher numbers common in conventional plate assays and powers of 2 and 10 frequently used or approximated in biological or chemical science or physics or electronics.

Error correction or other considerations may guide software to weight each sensor to meet a predetermined standard or may ignore output from one or more sensors. When connected to multiple elements, the output averaging output signals from each, connection of each element is optionally modulated to alter weightings of elements in the average. With fluctuating or non-constant inputs weighting is also controllable. For example, in an extreme sense a stochastic or alternating input, when alternated to off that element's output will report a zero weighting, or a fluctuating or stochastic feed can serve to physically, rather than mathematically control the weighting output. The designer and/or operator will have options for mathematical/algorithmic or physical/electrical weighting of each sensor element input to the data analysis. A group of elements may therefore receive the same feeder voltage, or the feeders may be independently controlled.

Instruction to or control of the system may be through information encoded on a sample package, information encoded on a sensor chip, from a user interface, information provided remotely by machine or active user, or information encoded within the device. For example, samples may be encoded with a shape or mass signal. i.e., a sample having a given shape would instruct the device to proceed with the assay that the software associates with that shape. In addition to shape, sample cartridge mass may be instructive as to the sample mass itself or may, perhaps distinguishing a smaller or a larger sample, instruct processing of the sample to allow access at controlled volume or feed rate of the compounds into analysis. An optically readable signal, (color, transparency, bar code, text, etc.) an electronically accessible signal (RFID, memory chip or drive, etc.), a magnetic signal, etc., are also usable in controlling the device. Specific control can be through a large variety of means and is not generally to be considered as limiting the invention. The signal embedded itself may be adequate to program the relevant machine cycles or may instruct the machine to access further instructions for example, in machine archives or at a remote location. A device may cycle through one or a plurality of signals as directed or required. Chips may be interchangeable and be encoded using signals analogous to those discussed above relating to sample cartridges.

A remote location providing instruction, collection, processing, and/or storage of data may be proximal to the device, i.e., in the same building, or may be distant, e.g., an unknown location in the cloud. Instructions may be self-sufficient or may query for further input from an operator or a distant database. Remote instructions may be signaled for production and delivery, for example, when the device is actuated by turning on or introducing a sample. Instructions may be stored in an arbitrary location such as the cloud which the device queries for specific operational signals. Remote signaling may be updated in accordance with experience of sister devices which can involve a neuro-like network. The remote instructions may be of any form, for example, explicit temporal instructions relating to each controllable variable, or more simply, to instruct to initiate one or a series of protocol apps available to the machine. For security purposes the device may require identification such as an access card, access code, facial or bio-recognition, etc. The device may be configured in many formats, e.g., as a portable point of care device, a mobile high throughput application, a fixed regional installation for massive scalable testing, etc.

Organization of Sensor Elements

The sensor elements are preferably nano-sensor elements to minimize size and maximize sensitivity of the sensing chip. Sensor elements will in general be mounted or carried on a substrate or support matrix forming a “chip”. Individual matrices may feature multiple elements, generally 10 or more, 32 or more, 50 or more, or larger populations of elements on a single chip. Individual matrices may feature elements with multiple different sensitivities including sensitivities for both VOCs and larger molecules on the same chip. As a rule, a greater number on a chip promotes a compactness desired for minimizing weight and size. The number of sensor elements is a design feature and can mimic numbers familiar to the operator or data analyst. For example, multiple of the number of wells common on petri dishes may facilitate using existing software tools to further analyze and compare results. Powers of ten, multiples of a hundred or thousand, powers of two are in common use. Accordingly, about 96 elements, 100 elements, 128 elements, 144 elements, 200 or 256 elements, 500 or 512 elements, 10³ or 2¹⁰ elements, 10⁴, 2²⁰, 10⁶, etc. may be built in as common useful working populations even if several elements on the chip are not activated. Sensor elements may be configured in any acceptable or conventional format, e.g., on disc shaped supports with modules sited at one or more radii from the center (to allow for spinning in a reader), on an elongated band,strip or ribbon (to allow linear advancement for reading), etc.

Minor variances in similar or analogous sensor sensitivities may be weighted internally by the machine software or may be overcome by averaging signals of a subpopulation of analogous sensors or chips. This massaging feature would be available as a tool to promote inter-device and/or inter-chip consistency.

Nano-sensor elements carried on the chips can be any properly designed sensing surface capable of, for example, field-effect transistor (FET) or other physico-electrical property/activity including, but not limited to: semi-conducting nano-wires, carbon nano-tubes—including single-wall carbon nano-tubes, chitosan-cantilever based, synthetic polymers—including dendrimers, plasmon resonance nano-sensors, Forster resonance energy transfer nano-sensors, vibrational phonon nano-sensors, optical emitting, optical frequency (or wavelength) based nano-sensors (sensitive to photon transmittance, absorption, reflection, energy modulation, etc.). Nano FETs and other nano-sensor formats generally operate by changing electrical properties as a substance comes in close proximity to the sensor by perturbing the steady state (absent the proximal substance) charges and movements (distribution of electrons) within the nano-sensor. When the transistor effective electrical properties cause an observable change in electron flow (current) this manifestation is one example of sensor competence. The altered distribution of electrons, depending on the design of the nano-sensor, changes one or more electrical properties, e.g., impedance, resistance-conductivity, capacitance, inductance, etc. and thus the physical movement of a detectable particle, e.g., an electron, a photon, etc. The present invention primarily features nano-sensors whose characteristics change depending on association (close proximity with) a chemical substance. Sensation may involve more than one event. For example, in one format of nano-sensor the proximity event may dampen a vibration that is sensed by observing a changed electrical property. Similarly, an optical property, e.g., reflectance, transmittance, refractive index, can be perturbed by proximity to a substance, altering electron distribution within the sensor enough to cause optically detectable geometric changes. The optically related detection format for a nano-sensor may be observed at a specific X or range of frequencies, e.g., moving peak transmittance to another X.

Categories of D: diode and T: Transistor nanodevices

| | | Operating | RS (resistance | | |
|---|---|---|---|---|---|
| Principle | Junction | Environment | sensitive) | Single Electron | Multi-electron |
| Field Emission | Surface | vacuum | dielectric field | single nano-tip | multi-nano-tip |
| Nanodevice | junction | | emission | emission; single | emission; field |
| | transmitter | | | photon devices | emission display D |
| Molecular | molecular | weak electric | molecular film | molecular wires | molecular rectifier |
| Nanodevice | switch; | interaction | sensor | MOS | D; molecular |
| | molecular | | | | tunneling D; |
| | light | | | | proton membrane |
| | | | | | device |
| Quantum | Joseph | low | coulombs | single electron | resonant tunneling |
| Nanodevice | device | temperature | blocker | devices; | tubes; nano-spin D |
| | | | | quantum spin T | |
| Semiconductor | Schottky SJT; | room | nanowire | single nanowire | multi-nano-wire |
| Nanodevice | surface | temperature | sensors; | MOS; particle | MOS; thin films T; |
| | plasma | | photovoltaic | electrometer | quantum dot |
| | | | devices | | memory cell |

A non-interactive or inert layer or zone may be disposed between sensing surfaces. Such layer may be disposed between decorated sensing layers in any pre-selected or pre-designed order, e.g., sensing layer based on graphene, decorated SWNT, inert graphene, decorated SWNT, inert graphene, undecorated SWNT, decorated SWNT, decorated SWNT, and the like.

Sensing elements in layers may be configured to form a tube of sensing layers arranged as a spiral. One embodiment presents as a plurality of bands or cord like structures. For example, in a spiral configuration, one or more relatively inert cords, e.g., inert graphene, undecorated SWNT, etc., is spooled in conjunction with sensing cords comprising graphene and/or SWNT. Inert cords may be incorporated as spacers to comport with design needs. For example, a triplet of inert cording may interpose between a sensing graphene cord and a SWNT cord, while a duplex inert cording may interpose between the SWNT and the next turn or the sensing graphene. The wrapped arrangement of the spiraling sensor bands may be of any desired formation, e.g., a tube arrangement a conical arrangement, an hourglass arrangement etc. The cord interior may carry the inputs and/or outputs for the sensors. One or more separate cords may be included in the windings to provide 10 capacity. The tube, cone, etc., may be wound about a framework that provides structural support as well as 10.

Another embodiment featuring spiraling architecture appears as a planar support, similar to a ribbon, preferably carrying sensing elements on both flat surfaces. When the flattened planar structure is twisted a spiral is formed. A linear arrangement has gases contacting the sensing strip from one end and continuing to flow along the plane,either flattened or twisted. Enclosing the band in a tube will allow gases to flow along the sensing band when one end of the tube (exhaust) is at a lower pressure than the other. Alternating pressures can produce alternating flows and more opportunities for sensors to associate with the VOCs. A twisted or spiraling band requires gas flow to bend around the spiral surfaces. In a closed tube or chamber rotating the outer wall or the sensing band increases contact incidents between the sensors and molecules n the gases in the enclosure. Inputs and outputs are not necessarily separated linearly, e.g., using a straight pipe or tube. The chamber may be curved, bent or shaped when desired by design features. The diameter may be varied along the length resulting in different linear speed of the gas molecules flowing through. The time of interaction between a sensor element and a molecule at different speeds can be used as a factor for differentiating VOC components.

The chamber may be continuous with no real termination, e.g., a circular or squashed shape and where gases may be introduced or exhausted at an entry and an exhaust port or through a plurality of ports along the flow course. Exhaust may be fractional. A portion of the gases flowing by a port may be removed while another portion remains in the flow. Exhausted gases may optionally be pressurized for reintroduction into the chamber to maintain the gas flux.

The sensor elements themselves or at least portions of the device surrounding the chips are preferably surrounded by a controlled gaseous atmosphere, generally slightly above ambient pressure to provide a slight clean room effect. The sensing chamber itself may have a reduced pressure with respect to the sample introduction area. A positive device pressure at at least one level surrounding the assay chamber is generally preferred to minimize possibility of contaminating inflows. The actual pressure where sensing is accomplished however can be varied. VOCs may be delivered by having a negative relative pressure in the chip area with respect to a sample containment or introduction area to cause drawing in sample off-gas when the off-gas collection volume and the sensing volume become connected. VOCs may arise from liquids introduced into the device whose components may have been or are being characterized by other sensor portions. VOCs may arise from samples analyzed by VOCs themselves, for example, when heated may produce the pressure difference to drive delivery to the sensor volume.

Since physical delivery or movement is required to bring a candidate compound in contact with a sensor element, a physical intervention is required. Physical movement can be induced as desired by any appropriate force. Forces may be constant, variable, stepped or pulsed, etc. Multiple forces may be used in series or parallel for sample delivery or a single force may be selected from the device's repertoire to enhance delivery and detection of the sample to the sensor element(s).

For example, temperature can control speed and movement of target compounds and ambient gases driving the sample compounds to the sensor; pressure difference can induce a convective movement. Pressured gas canisters may provide a driving force. Other forces including, but not limited to: electric, magnetic, electromagnetic, acoustic, photo-excitation, spray, or photon momentum, etc., may be selected depending on particular circumstance. Forces may be described in a number of ways. In one example, a decrease in temperature may induce a relative vacuum thereby creating a convective force. An acoustic force, for example, having one or more oscillating frequencies in a range perhaps between 10 mHz and 100 MHz will often exhibit one or more harmonics (or multiple frequencies). Echoes may result in one or more frequencies that are distinct from the feeder frequency. Geometry and chemical composition of the device may accentuate or dampen frequencies. The acoustic engineer will take into account the importance of such effects when designing the device.

The gaseous environment in the present invention is an improvement over prior applications of FET sensing in that the response is both quicker and reversible. Reversibility is critical for high-throughput commercial applications in that it allows for the rapid turnover of samples through avoidance of disassembly and/or cleaning between sample readings. The sensor elements on a chip are thus available to assay hundreds or thousands of samples in a day. Reversibility can be accomplished simply by increasing the temperature. Flushing with the ambient gas or another gas can also be used. Continuing to monitor the output signaling from the sensor elements provides assurance that the sample has been reversibly cleared and the device is in a mode to accept the next sample. Graphene based sensors exposed to liquid may become compromised following many assay exposures. The graphene based liquid sensing layer may be misted with water or salt solution followed by water, then a flush with ambient or inert gas such as nitrogen. A more rigorous restoration can be periodically accomplished, e.g., as every 5^{th}, 10^{th}, 20^{th}, or other practical rate includes isopropyl alcohol, for example, between 10 to 50%. Ethanol may be substituted for isopropyl at 1.3 to 1.5 times the amount by volume.

The gases or atmosphere surrounding the sensors will comprise molecules that interact with sensor elements to—after signal generation, transduction and processing—output information relating to the components in the vapor from the sample being examined. Pressure within the testing chamber is preferably maintained by controllably providing a non-reactive or inert gas. Argon is such a gas often used in manufacture and medical applications, and helium, nitrogen, neon and xenon may also commonly meet the needs of non-reactive or inert applications. Some applications may suggest using a mixture of gases maintained at conditions compatible with testing. For example an extremely light gas such as hydrogen or extremely dense gas such as tungsten hexafluoride may be the only gas used in certain applications or may be admixed to arrive at desired properties, including, but not limited to: acoustic, temperature, reactivity, shielding, polarization, viscosity, etc. The specific gases used for assaying samples comprise one controllable variable that may be maintained as constant or varied during an individual sample reading.

At least one, but often a plurality of sensor chips, may be included in a device. During use, the sensor chips will be mounted in a controlled atmosphere chamber where vapor phase analyte will be introduced to contact with the sensor chips and thus the sensor elements. During analysis, input and output voltages are provided and monitored, respectively, as analyte is delivered to the ambient volume over the chip. In several embodiments, only a vapor phase analyte contacts the sensor elements. This provides advantages over many liquid phase SWNT and similar sensors in that sensor size can be reduced without having to account for surface tension, liquid phase excipients are not necessary and turnover rate is not compromised by the requirement to remove the liquid carrier. Where liquids are delivered into the device, it may be preferable to include replaceable or disposable sensor modules or chips that may be serially or sequentially spaced, e.g., on a band or ribbon or a line of chips that may be delivered into the device as their turn arises.

Sample Suitability

For medical applications, the analyte sample is most preferably a non-invasive, readily available, biopsied sample, though in some applications breath or ambient air may be collected. Urine is an excellent biopsy sample, given that urine includes filtrate from the entire body and is non-invasively obtainable. Urine is especially preferred in that it is a sterile medium with no known shedding of SARS-CoV-2 virus or particulates. Urine is also easily collected using sterile technique without any specialized training. However, in specific cases a biopsy from any tissue may be analyzed. As a useful contrast, earwax is easily obtained and may be used as a biosample. However earwax is not sterile and includes microbiome components (Microbiome components are not necessarily less desired. A number of microbiomes are critical for a healthy organism.) Non-urine biopsies may involve additional preparation such as cell lysis, centrifugation, washing, extraction, etc.

A feeder cartridge is often used to present the sample. The present invention is not limited to any specific feeding protocol. For example, the device may be fed manually, by a machine carousel, by a linear feed, by a high rate delivering robotic system, etc. In one preferred embodiment the cartridge in lined up in a queue for delivery to the device. The cartridge can bear identification allowing tracing of the sample through data collection, signal transduction up through final output. Several analytical processes may be chosen. The cartridge may contain or support the sample in any suitable presentation format, including, but not limited to: in a pierceable liquid container, an open top container, a container sporting an access port, a charged gas cartridge or gas cartridge with other ability for expelling a sample, a spongy material (such as a synthetic plastic or foam or a natural material such as a fiber like cotton) for presenting the sample, a dried or lyophilized sample for reconstitution, a frozen sample, a piston device (e.g., a syringe), etc. In especially elegant applications a photon momentum force can be applied in an atomic tweezer fashion. Selectivity can be further enhanced by tuning the light frequency to optimize retention or exclusion of different compound species.

Physical Format of Device

One example, just illustrative in nature, comprises SWNT nano-sensor chip that is positioned at the top of a chamber with graphene, liquid sensing chip(s) below. In this format as with any format, the chamber may incorporate one or more auxiliary sensors, e.g., a temperature sensor, a pressure sensor, etc. A tray for transporting and delivering a sample or samples may be removable and replaceable, slidably accessible, accessible through an openable or removable port, door, cover window, etc. Retractable and/or replaceable panels may be slidably inserted in the device to provide solid or perforated barricade where separation/isolation or elemental directed access is provided.

In a subset of embodiments, a second slidable panel may employ offset perforations to control access allowing convective access of analyte to elements in an open position and shutting off access in a closed position. A heat source, such as a heater plate may be set to a static excitation temperature to help excite the VOCs or may be varied, for example for ramping or cycling to control vapor pressure of different classes of components in the sample(s). Temperature may be controlled by a heater in contact or close proximity to the chip or through controlling ambient gas temperature. Each element on the chip may be heated individually when heater elements, e.g., resistive circuits, are placed in conjunction with the sensor element. Device embodiments may enable, and/or method embodiments may use: temperature, electromagnetic stimulation, physical stimulation, chemical stimulation, etc. to deliver or modify sample product for analysis.

In some embodiments, a liquid sample is in a well of the device. The device comprises an elevator with controls capable of raising the sample such that liquid immerses a lower ntG layer and leaves an upper SWNT sensor layer in a gas sensing environment. Contact between the liquid and ntG sensor layer may be sensed using a signal, including, but not limited to: laser, thermometer, sensor activity, ultrasound, etc. The first and second sensor layers may be independently controlled or controlled as a unit. The elevator system may lower sensors mounted on a rod, shaft, or column or may raise a sample reservoir. A sensing shaft/column is lowered such that a ntG sensor layer is immersed in the liquid. Preferably the shaft is heated to help volatilize compounds in the liquid so that they rise from the liquid and are available for assay by one or more SWNT layers. A heating element in the shaft or sample reservoir container preferably augments a vaporization process. Contactless heating can be accomplished using electromagnetic, ultrasonic, or convective excitation. An alternative embodiment has the well heated. In some embodiments, the well may be elevated rather than the shaft lowered.

The shaft can be hollow, similar to a straw, or solid. Sensors can be displayed on the inner or outer surface with the first layer (ntG) positioned for initial contact with the sample. A hollow shaft may draw liquid into the interior to contact the ntG sensors while the SWNT sensors are disposed outside the shaft or higher up in the interior. A similar embodiment features ntG sensors on the outer surface while a limited diameter and or a pressure in the shaft allows only gas phase molecules to enter.

The elevation system also supports embodiments that assay a solid biopsy sample. The sample is staged on a platform. The sample is optionally heated. The sample is elevated or the array(s) lowered to bring the sensor module in contact with the sample. Liquid from the sample contacts a ntG sensing surface. This surface is porous to VOCs to allow access to SWNT sensors. The SWNT sensors may be mounted on a surface of the chip opposite the ntG sensors or on an adjacent support.

Operation of the Device

Depending on delivery method and time of residence in the assay chamber, a sampling rate of less than 1/min is currently achievable, including misting and flushing with gas. Sampling rates of about 1/sec per sample are possible in the gas only operations. Longer sampling periods, perhaps up to 10 to 20 minutes, may be used during optimization of a newer testing protocol. The speed logistics may be impacted by the rate at which samples can be collected and delivered. Flushing or replacing used chips may be performed in an accessory component that is inserted as a unit for the next sample analysis.

Pressure differential, released gas, vibration and sonication are common formats for physically stimulating and moving a sample. Electromagnetic stimulation may comprise visible or non-visible wavelengths. Laser stimulation allows fine control of intensity and targeting. Physical and/or electromagnetic stimulation may modulate temperature; resistive heat coils, hot plates and liquid radiators are additional options for heating or chilling a sample. Forces including, but not limited to those of: photo momentum, acoustic, gas flow, magnetic, electromagnet, electric, Lorentz force, chip replacement, etc., effects can be used to control movement of sample compounds.

Stimulation may be constant, may be ramped, or may be paused, pulsed or increased in time or intensity as the analysis requires. Changes may be linear or non-linear, and in practice can be programmed to any desired pattern. Multiple stimulation types may be used in parallel or in series and may be pulsed or varied between types—with each type independently controlled. In some analyses, stimulation for a desired time/intensity may be employed to flush, for example, high concentration volatile components, from the sample or to fragment or combine sample constituents.

Sensor Elements on the Chip

A chip may be formed in any desired configuration. For example, a 10×10 sensor array on a chip can provide a compact yet exuberant surface. Arrays may be constructed to align with squares or powers of 2 as is common in computation devices and some biological plates. Thus, for some embodiments a 2×2 sensor chip may be sufficient. But more often a greater number of chips will be employed for additional sensitivity and discrimination abilities allowing assay results to be collected on a greater number of analyte chemicals. Thus, a 3×3, 4×4, 5×5, 6×6, 8×8, 10×10, 12×12, 15×15, 16×16, 18×18, 20×20, 25×25, and so on, including intervening squares, mentioned and envisioned here, but not exhaustively incorporated in the text format might be constructed. Other non-square formats are also envisioned. In biology plate sizes based on a power of 2 times 3 are often employed. Thus 48 well, 96, well plates, etc. are common and easily handled by modular software applications. Since binary electronic electronics often increase capacity according to powers of 2, but physical dimensions may not always be supportive of such doubling with each improved version. Software may often be capable of addressing a number in excess of the sensor elements on a chip. For example, computations relating to 2⁶ may be used with a 7×7, 8×7, or 50 element chip. A 10×10, i.e., a one-hundred element chip may be served by an application designed for up to 2⁷ (128) element channels. Higher element chips may thus suggest using applications that have capacity for 2⁸, 2⁹, 2¹⁰, 2¹¹, 2¹², 2¹³, 2¹⁴, 2¹⁵, 2¹⁶, 2¹⁷, 2¹⁸, 2¹⁹, 2²⁰, etc., sensor element channels. The chip may be configured with one dimension far in excess of the other. For additional capacity or perhaps to consolidate assay functions in between bulk restoration functions, a chip may be confi-gured in a flexible format in the form of a ribbon with a fraction of the length presented at each analysis function. For example, several 12×8, 10×10, 12×12, 20×20, etc., analytical portions might be exposed with the ribbon being advanced for each subsequent analysis round. A mask may be used to expose only a portion of the chip, for example to select a class or classes of decorations for different exposures to sample VOCs. Such mask may be perforated and movable allowing multiple reads on a portion of the chip without need to restore or advance the chip after each assay or assay condition. A mask with perforations can be made to allow for multiple sensor layers to reside on a chip. For extra high throughput, the ribbon may be advanced in continuous movement where samples are presented at a high rate, perhaps 1/sec. The just used portions of the ribbons may be restored in series with assays by passing through a restoration chamber or may be constructed as a disposable cartridge.

A restoration or cleansing step associated with each analytical round might involve a continuous cartridge or band, where, for example, the assay is accomplished in the assay chamber and when the band is advanced, it passes through another chamber, possibly with a different ambient gas at a different temperature. The chip temperature might or might not itself be specifically or self-controlled in this format; the ambient gas could provide the thermal energy releasing the assayed compound from the sensor element. An atmosphere different from that in the sampling chamber might be used in the restoration phase. Bulk restoration, for example, at the end of a shift might be desired in some circumstances.

The ribbon may be a rigid or semi-rigid strip or might be flexible so as to be compactly spooled. As costs of the sensor elements decrease, ribbon formats may be desired and permit disposal after use.

Thin sensor elements are preferably extremely compact in size to permit high density and smaller device footprint. Sensor elements on a chip will be separated from neighbors by insulation barricades. Many insulators are known and can be selected during design based on parameters such as appearance, size, cost, assured availability, etc. Polyamides are common inexpensive insulation barricades. Circuit board material (e.g., FR-4, CEM-1, CEM-3, RF-35, halocarbons, fluorocarbons, Teflon®, PTFE, polyimide, etc.) are also strong candidates for use in high production models. Depending on material and anticipated voltages, an inter-element separation of ˜50 nanometers (nm) is often sufficient. Larger voltages may require greater isolation distance. The elements themselves may be any desired shape, e.g., rectangular, rhomboid, hexagonal, triangular, elliptical, circular, irregular, creviced, crumpled, shredded, perforated, layered, masked, etc. Sizes can be miniature, e.g., ˜40-50 nm thus suggesting the term nano-sensor. Circuit board materials known in the art and potentially usable as substrate or laminate in embodiments of the present invention include but are not limited to: FR-4, FR-4 High Tg, RF-35, Teflon, CEM-1, CEM-3, Polyimide, PTFE, etc.

Size is a simple design consideration involving e.g., manufacturing efficiency, device dimensions, density of sensors, surface to volume ratio of NSE, sensitivity of detection, durability, cost, etc. Accordingly, sizes of elements may be in the area of for example, 40 nm, 50 nm, 75 nm, 100 nm, 200 nm, 250 nm, 500 nm, 0.5 μm, 1 μm, 5 μm, 10 μm, 50 μm, 100 μm. Shapes may be planar, essentially flat on the substrate surface, or at an angle disposed off the surface. Shapes may be irregular, e.g., crumpled or creviced. Shapes may be regular, e.g., hexagonal, creviced, etc.

In accordance with this invention, the conical geometry of the sensor chip provides an improved contact interaction zone, by increasing surface area and variability of the sensing portions while also reducing the distance a VOC compound must traverse for multiple interactions. The device provides more interactive opportunities between molecule and sensor element and in several embodiments interactive opportunities for larger complexes or compounds that may themselves not be VOCs.

A sensor element disposed on and above a substrate surface may be designed as such to increase or maximize surface area to interact with the vapor that may or may not include a VOC of interest to said sensor. The present invention features pyramidal or pointed shaped projections rising off the substrate surface. As an example, an array of regularly spaced sensor elements arranged in a 16×16 pattern results in a 256 sensor array. Other examples may include 20×20 (400 sensors in array), 32×32 (1024 sensors in array), 64×64 (4096 sensors in array), etc. Arrays however are not limited to square dimension, e.g., an array might be formatted as a string, e.g., a 1×100, 1×1000, 1×10,000, 1×100,000 line, a narrow width, e.g., 2, 3, 4, 5, 6, 7, etc. width with a 3, 4, 5, 6, 7, 8 length, respectively. The difference between length and width may be 1, 2, 3, 4, 5, 6, 7, 8, etc., or designer selected multiples including, but not limited to: 2, 3, 4, 5, 6, 7, 8, 10, 12, 15, 20, along with fractional multiples that produce integer values. Using the 256 sensor array as an example: A first row (16 sensors) may be constructed with a first decoration, e.g., a nucleic acid having a manageable number of bases that are easy to accurately reproduce for binding to the carbon support layer. Second and subsequent rows may be configured with a different functionalizing decoration unique to each row. In this example, row and column are used arbitrarily to apply to two dimensions on a surface. The number of rows need not equal the number of columns. This is a design choice. For example, when sensors are configured on a spool or ribbon, the width will sport much fewer sensors than the length.

Nucleic acid functionalizing a first row may be similar to a different nucleic acid used to functionalize a second row, for example a first functionalizing DNA may be a 15-mer that comprises a 9-mer of a second row functionalizing DNA. Functionalizing compounds are not restricted to DNA. However, DNA is a preferred substance because of its organic structure that coordinates with the carbon support and volatile organic carbons. Lengths preferably are in a range inclusive of 8, 9, 10, 11, 12, 13, 14, 15, 16, 17, and 18. But longer lengths, e.g., 20 to 24, 30 or 32, are appropriate when tighter coordination may be desired.

In one elongated format where length is several times greater than width, strand or ribbon shaped embodiments are crafted. In one embodiment a ribbon shaped (flat strip) is formed into a spiraling twist. Sensors may be periodically disposed along the length, at one end, in the middle, or the entire length. When confined in a tube the ribbon may be rotated such that gases are propelled along the length. In some embodiments an external gas driver provides propulsion. In another format, the twisted ribbon is disposed in a circular tube. Gas may be driven several cycles through the tube to multiply potential interactions between sensors along the length. In these embodiments, the rows may be disposed across the ribbon and in some embodiments, multiple rows will sport identically functionalized sensors.

Sensor elements will generally be supported on a non-conductive substrate like Si or synthetic polymer. The spike shaped structures of the present invention may be grown on a base support that features circuitry spot connecting to each projected sensor site or locus. A pyramidal sensor is disposed or manufactured atop each active locus. (A chip may comprise a higher density of terminal loci than used for sensor disposition. The designer may find that economies of production may suggest a bulk acquisition of high density chips with multiple terminals ignored or unpowered.)

One embodiment features constructing a spike or spire of carbon e.g., a SWNT. These may be printed or deposited in multiple pulses of e.g., liquid carrying the SWNT to build a spike rising off the terminal. A metallic type SWNT acts more efficiently as a conductor. Semiconducting formats including but not limited to those mentioned above are available as spike support materials. The spire serves as a skeletal structure for a multiple application grown pyramid. The spire may have a shape with the base approximately identical in diameter to the diameter near the top. The spire may be tapered, e.g., with the distal portion of the spire having a diameter about: 0.1, 0.2, 0.3, 0.4, 0.5, 0.6, 0.7, 0.8, 0.9, 1.1, 1.2, 1.3, 1.4, etc., times the diameter at the base. The spire need not be pole shaped. It may be configured as a slightly rounded wall or a planar structure rising from the base at an angle greater than about 30 degrees, and commonly greater than about 60 degrees. An angle of spire to base of about 90 degrees or approximating a right angle may be preferred in many embodiments.

Choosing a solvent appropriate in felicity to the spike, a first ring of carbon nanotube sensor base is laid at the spike bottom. A decoration solution is applied as a second layer. An inert layer optionally is layered between one or more, and possibly each, decorated carbon tube sensing layer. Subsequent carbon tube containing solutions optionally comprise a removable filler. The filler is ablated, dissolved, evaporated, or sublimed out creating a porous structure. Preferably, 3, 4, 5, or 6 bands of decorates SWNTs are ringed in pyramidal formation.

A second embodiment eschews the spike. The pyramid is principally built by layering. A base nanotube layer is applied, then decorated. A second nanotube layer is applied atop the first but with a smaller volume of nanotube to form a smaller diameter layer which is decorated. A third, fourth, fifth, sixth, seventh, eighth, and subsequent layers are applied in accordance with the designers instruction.

The ratio of pyramid height to base ring diameter is a design choice, e.g., about 0.5, 1, 2, 3, 4, 5, etc. Spacing between bases is preferably compact to minimize device footprint. Spacing must be compatible with the circuitry but may be negligible or any distance allowing the sputtering, dispensing, or printing to be accurately practiced.

In general, each layer of a pyramid will sport the same functionalizing compound. However, functionalization may be omitted, leaving a later minimally attractive to VOCs to concentrate interaction with decorated layers. For example, every other or every third carbon layer may be left undecorated and thus relatively inert.

In the 16×16 example mentioned above, 16 different decorations might be applied to the 16 rows. The 16 columns then act as “duplicate”, backup, or confirmatory sensors, whose outputs are accounted for in the algorithms used for running analyses. Row elements are not necessarily identical. “Duplicates” may be split along a column, e.g., a half, or quarter of the length. Duplicated may be blocked, e.g., in the 16×16 example, as 4×4 blocks. In a 16×16 sensor chip, for example, each row may have two different decorations, resulting in 32 different sensing types if all rows are split.

In some embodiments dead zones are effective for improving sensitivity and reliability. Similar to the embodiments where individual non-decorated layers are used as spacers, Undecorated (non-functionalized) SWNT towers or pyramids are alternated with functionalized zones. Though generally treated as inert, in some embodiments these non-functionalized “sensor” towers may be included in the circuitry as machine diagnostic or control sensors.

VOCs in Contact with the Chip Sensor Elements

As designed, the VOC sensor element portions do not contact the non-gaseous sample itself, only vapor or gaseous phase emissions off the sample are available for interactive transient contact. Vapor constituents, as the vapor flushes through the sampling chamber, will transiently specifically non-covalently bind to or associate with one or more of the decorations incorporated on the sensor elements. Depending on temperature and possible gas or combination of gases used as ambient atmosphere, the collated sensor element data produce a characteristic response for a specific VOC, combination of VOCs, or class of VOCs. A chip generally will be coated with several types of sensor elements whose sensing specificities are distinguished by having different decorations such as nucleic acids attached on their surface. Polyaromatics and aromatic peptides, including synthetics, may also be valuable decorations. Differential specificity of a chip element may be exhibited at different temperatures, in different atmospheres and/or in different sequence patterns of exposure.

Temperature is significant for at least three important reasons. One, at higher temperatures, the molecules will have higher kinetic energy and thus be less likely to sit docilely on a surface. Dif-ferent VOC compounds will exhibit different specific temperature effects as will different decorations. Two, the actual VOC chemical may tautomerize or morph, perhaps with a higher temperature favoring a different bonding structure than a lower temperature. A compound might then, in some embodiments, be detected on different sensor elements at different temperatures. Such assay response may be used to more specifically identify and assay a particular compound. Three, improper temperature management can comprise sample integrity, for example, leading to clumping, particulate contamination or other sample degradation.

A sample may be introduced into the testing chamber and maintained therein while a change in temperature alters the sensing specificities of the elements on the chip(s). In embodiments where individual sensors have dedicated temperature controls, sensor elements with identical decoration, but read at different temperatures can provide the temperature differentiation analysis more rapidly, i.e., without waiting for a temperature change on the entire chip thus aiding in high throughput analysis.

In general, the interactions between the sensing portions of each sensor and the sensed analyte are low energy bondings or coordination complexes between organic molecules. Bonds do not involve covalent reactions and thus are reversible by changing the conditions in the chamber. Dilution, i.e., simply flushing, in many circumstances will ready the sensor element for its next round. Optionally, a different gas is used for flushing and/or a higher temperature may be used. In addition to thermal or convective restoration processes, any format used to physically move the compounds can be used alone or in concert with others to excite/remove the panoply of complexed VOCs. Forces including, but not limited to those of: photo momentum, acoustic, convective gas flow, magnetic, electromagnet, electric, Lorentz force, chip replacement, etc., effects can be used to prepare for a subsequent sample read. Non-convective restorative forces will be especially advantageous in low pressure, or no added gas embodiments. The broad spectrum of restoration options enables testing of multiple samples types in high throughput operations.

### EXAMPLES

On a chip, a simple configuration in binary format comprises an element grid arranged in a 16×16 (2⁴×2⁴) pattern, i.e., 216 (2⁸) elements. Larger chips generally but not necessarily may follow a continuing binary pattern, i.e., 32×32 (1024 or 2¹⁰), 64×64 (4096 or 2¹²), 128×128 (16,384 or 2¹⁴), 256×256 (65,536 or 2¹⁶), etc. A chip may not use all elements as active sensor elements. Some may be inactive, some may be held in reserve, some may serve as controls or calibration elements, etc. The chips are functionalized or “decorated” single wall nanotubules (SWNTs). Nucleic acid molecules are inexpensive decorations that can be made with thousands of options. Using non-natural, i.e., nucleic acids not in the human genome or RNA repertoire, many more specificities can be addressed. Amino acids with ringed structures can be incorporated as functional coordinating binders. Thus, specificities of sensor elements are tuned to the desired conditions. The identical decoration can display different specificities as temperatures change.

A base voltage of generally is in a relatively low, i.e., non-arcing or insulator damaging range for example around 1 pV, but more normally up to 20 V, is applied to an input electrode of a sensing element. 10⁻¹⁸ amp is a minimum amount sensitivity with 0.4 fA being characteristic of our current implementation. These values may improve with experience. The voltage may be static or oscillate (either deterministically or stochastically). Oscillation may include ranging from positive to negative voltages, may include simple on-off switching or other square wave pattern, saw tooth pattern, triangle pattern, stochastic, etc. Voltages may be stepped through a range or introduced in a ramping or cyclic (e.g., sinusoidal) pattern or stochastic perturbation. Voltage may be sent to each sensing element individually or the same voltage may be applied to several sensors, including circumstances where all sensors are fed identical input.

In the sensor element, current is or is not delivered from an input electrode to a corresponding output electrode through a field effect transistor carbon layer. In one set of examples the carbon layer is formed as a single walled carbon nanotube (SWNT) layer. In the on mode, the SWNT carbon conducts a current through to an output electrode. When the field effect transistor is in the off mode, the current does not conduct. Several such elements are attached to form a nano-sensor chip. The conductance of the SWNTs on the elemental surface is perturbed by close association with a target compound, for example a volatile organic compound. Binding of such target compound modifies conductance of the SWNTs in such a fashion that the coordination binding acts as a transistor switch turning the gate on or off. In some instances, the coordination will be probabilistic with rapid gating as different portions of the target compound may bind to the SWNT, perhaps at slightly different coordinating atoms. Such probabilistic binding may be temperature or voltage dependent or may vary with the delivering gas. In other instances, the binding may be more constant, simply gating for a range of temperatures/voltages with large zones of on or off signaling.

Specificity

Specificity of coordination is provided by functionalizing or decorating the carbon gate electrode. For example, many sequences of nucleic acid such as DNA or RNA will stringently coordinate or bind with the SWNT structure. These nucleic acids may comprise naturally occurring bases, e.g., those based on :adenine (A), cytosine (C), guanine (G), thymine (T), or uracil (U), or analogous or synthetic bases. The ringed structures of the nucleic acids or other molecules such as peptides containing a large fraction of ringed structures associate strongly with the nanotubular structures. These functionalizing or decorating additions to the SWNTs serve to selectively capture proximal molecules. When the chemical geometry is thus changed, the gating characteristic of the associated carbon bridging the input and output electrodes is modulated. A single element may be associated with a single sequence or a plurality of functionalizing sequences. Output characteristics of gating in response to one or more gaseous compounds, e.g., VOCs are then collated into a data library. When that sensor element responds in the same manner, presence of the VOC is confirmed. Stringent selection of element functionalizations, and subsequent application of the controllable assay variables can optimize certainty of VOC identification at a desired level, for example, increasing manipulation of the variable parameters can achieve certainty of 99+%. In special circumstances, for example to develop rapid profiling of a new VOC signature (i.e., pathogen), a simplified screening protocol or developmental process may begin with a lower level of certainty, e.g., 85%, 95%, etc. Subsequent refinements then could be applied to raise the level of certainty until reaching a mathematical and chemical sensitivity to an acceptable level, e.g., a 99+% certainty.

A single element may be capable of indicating the presence of more than one compound. For example, similar compounds may not be distinguished in their association/coordination with the element surface and therefore may in certain circumstances produce indistinguishable signals on their own. But the single element may, for example, in conjunction with one or more other elements provide definitive results with respect to the VOCs that may interact with any one element. Alternatively, the single element when operated at a different temperature, voltage or other variable may distinguish between the different compounds binding the element under static conditions. The discussion above describing the variable inputs and input patterns and different resulting outputs relates to such differentiation capabilities.

Fast Tracked Testing and Reading

One embodiment may include a simplified assay, perhaps a chip with fewer component element or element types, e.g., using only a fraction of the DNA species on the general use chip. In simplified embodiments fewer parameters may be manipulated, perhaps a static system where one or more variables, such as, voltage, temperature, etc. have a reduced range or remain constant. When AI identifies, for example, a simplified signature for a specific set of diseases or a specific disease, such as a new virus or strain of virus, the device may be instructed to operate in a simple detection mode similar to that of a +/−strip test. Chips may thus be made specific for different preferred assays or a regular chip may be used with simplified readings.

A simplified data analysis may be inherent in the chip. For example, a circuit can be built with specific sensors in series and/or in parallel. When the circuit produces the right gating, a positive result would be output. A side circuit on the chip possibly sharing portions of the positive negative circuitry may be included as a control. In some embodiments a completed control circuit with an incomplete or open positive circuit may produce a “negative” signal. The chip itself may contain a coded instruction for the machine to operate in the designated mode, e.g., an optical patch, physically slotting, an RFID, actual machine readable code, etc., may instruct the machine to operate in the preferred program manner. Such a streamlined approach can enable extremely high throughput analysis of targeted profiles.

Shielding

As sensitivity is heightened, machine stability becomes more important. Therefore, depending on output sensitivity targets, formats of samples, formats of delivering the samples, etc., shielding is considered a major design consideration. For example, if acoustics are used to advance, modify, present, or to remove samples, acoustic shielding in the relevant wavelengths and consideration of harmonics of the structural hardware, should be taken into account during design and installation. Passive, e.g., sound insulation, or active, e.g., sound cancellation shieldings are compatible with such shielding requirements. Electromagnetic shielding can be any suitable format, e.g., conductive material such as copper, nickel, mu-metal, conductive plastics, conductive paints/inks, etc. In general, the device should be protected or shielded from any influences, that interfere with performance including, but not limited to: acoustic, temperature/thermal, electromagnetic, visible, infrared, ultraviolet, radio/micro waves, magnetic, electric, etc. For particular environments, including, but not limited to: space travel, zero or low gravity, proximity severe weather events, deep sea or deep underground, high altitude, atmosphere, where the device is to be used, additional shielding, e.g., from heat, ultraviolet light, solar wind, ionizing radiation, high velocity transit, constrained environments, densely populated locations, proximity to nuclear power plants or engines, vibration, etc., is a desired design feature. While general ambient conditions for most of the device's intended uses will be relatively standard. When a device is designed for use in any extreme environment, additional relevant shieldings should be studied and applied where appropriate for example when designed for use for a long duration space flight.

Data Storage and Analysis

Raw data may be stored in a library linked to the sample source with any other relevant information including, but not limited to: disease diagnosed, disease status, nourishment history, time of collection, volume of sample, volume analyzed, medical history, preparation steps before analysis, storage and/or chain of custody conditions, medications, gender, age, etc. Such library may be stored or transmitted in any available format and process taking safety, privacy, consent, cost, relevant laws, legal jurisdiction, storage density, transit speed, etc. into account with a goal of interfacing groups of machines in a knowledge base where each device teaches and learns from others. Portions of the library may be stored in diverse locations including any available format, e.g., single encrypted, double encrypted, or block-chain coded.

Files in such library may be compiled and analyzed by knowledgeable humans, but more preferably using machine learning and/or artificial intelligence in any combination. Such processing, analysis and comparing multiple samples with associated information will then be useful for continuous expansion of the disease repertoire and the improvement of diagnostic accuracy and quality of the output data.

Early Warning System

One especially poignant application of this device and technology relates to infection by a virus. Viruses are often specific to a small population of cell types at a particular state of development. For example, in the case of a corona virus such as the SARS-CoV-2 virus that is responsible for the COVID-19 pandemic, the “spike” or S-protein binds to Angiotensin-Converting Enzyme 2 (ACE2) found on human cells. The spike protein also acts in conjunction with another cell surface protein, TMPRSS2, to initiate cell processes causing viral entry. ACE-2 is found on multiple cell types in the human body, including, but not limited to: endothelial cells of the circulatory system, enterocytes of the small intestine an in especially high numbers on Type II alveolar cells in the lung. Type II cells are the cells that secrete surfactant coating the air sac surfaces. Surfactant lowers the surface tension of the fluid coating the alveoli and thus helps to keep air sacs in an open, rather than a collapsed state. When the alveolar cells are targeted and eventually killed to release new virus, breathing becomes more difficult as the air sacs lose air exchange surface area and increase amount of fluid in the lung. This diminished lung function can be diminished further as the immune system gears up to fight off the virus. The immune responses can further fill lungs with fluid and pus and severely compromise breathing. In this example, the type II cells are known for high contents of dipalmitoylphosphatidylcholine, ethanolamine, cholesterol and many trace organics.

Certain adaptations of ACE2 bearing cells resulting to their adaptations to stresses from obesity, renal disease, cardiac stress, etc. apparently makes these cells better targets for the virus. Lung cells die in high numbers and release contents upon cell lysis and death. Some content is expired in the breath, but most is transported by the blood for processing and removal. When the blood is filtered by the kidneys, VOCs released during cell lysis will be delivered into the urine. The appearance of VOCs in patterns relating to a type II cell origination provides evidence of attack on these cells. Knowing which pathogens are in circulation can increase the certainty that the source is from the SARS-CoV-2 actively pandemic virus. The human, artificial intelligence, and/or machine learning can be more certain when more information is available. For example, persons with the adapted and compromised cells mentioned above will also exhibit breakdown products from these cells in their urine. Assays indicating breakdown products from the non-lung compromised cells can provide stronger certainty that the SARS-CoV-2 is the actual culprit.

Identification of Emerging Biothreats

However, even before a virus is identified, distinct patterns associated with an unknown disease may appear in several samples and be concentrated in one or more locations. This information may help to characterize or identify the unknown pathogen because, when cells are attacked and become infected, they start producing and releasing abnormal VOCs and abnormal levels of other VOCs characteristic of the cell type. Therefore, a network of devices deployed in hospitals (and military installations, factories, airports, laboratories, points of entry, etc.) can act as an early warning system of new emerging viral threats whether natural on man-made. Such system can rapidly identify and provide a VOC profile of the new pathogen suitable for use in identification of healthy or infected individuals even before genetically decoded and named. The present invention eliminates the need for genetic decoding testing and development, manufacture and distribution of new testing kits. The device of the present invention does not require special chemical analytical reagents or specially trained laboratory personnel and can be self-administered. When the preferred analyte, urine, is used, only minimal chemical or biolab protective gear is necessary. Therefore, massive scalable testing is enabled using urine (a sterile sample medium without intact pathogens) does not emit particles into the ambient environment.

Each active infective virus will induce specialized responses in the target cell thereby producing a signature pattern of cell metabolism that results in a VOC population highly associatable with the specific virus, sometimes even a specific strain of virus. Each virus requires a receptive cell to endocytose the viral genome and generate copies of new virus. To gain access the virus must bind to a particular portion on a cell. Viruses are thus unique in the cell type or cells types they attack. The pattern of cells and the responses to the attack will be unique to each new virus. So when devices of the invention are interfaced with each other and share data, an early warning wake-up call can identify a potential health risk long before it becomes a crisis or would be recognized following current practice. Through relation to previous data, the algorithms can identify the targeted cells and therefore potential anomalies and treatments related to the cells under attack.

Containment of Emerging Biothreats

Urine samples assayed using the present device can deliver a unique signature profile associated with the viral infection within seconds or minutes of sample introduction. Because the renal system filters blood from all body organs, urine contains metabolic products produced in each organ and therefore will contain VOCs characteristic of the cells under attack. This can help to identify the pathogen in some circumstances, but with new pathogens, by identifying the cells that the viruses use for replication can suggest who is at greater risk and can help in rapidly developing treatment protocols. The rapid identification and recognition of an emerging biothreat combined with massive scalable testing as provided for in the present (networked) invention will enable rapid containment of a geographically designated containment zone greatly reducing the virus' ability to continue its transmission.

Assays under the present invention can recognize a new appearance of previously known signatures or appearance of a previously unknown signature. Geographic location, possibly to a single town, lab, hospital, metropolitan area, zip code etc., will allow instant designation of proposed containment zones. In these zones, rapid testing of associated individuals and contact tracing where relevant can be initiated and accomplished before the numbers of afflicted persons become burdensome or overwhelming. An outbreak, even from a previously unknown pathogen, can therefore be identified and stopped long before reaching epidemic status and hence prevent an epidemic event expanding into a pandemic. In conjunction with the identification and partitioned follow-up of the disease making a first appearance, the disease can be characterized as to source, afflicted cells, possible treatments and/or preventions, etc. Each of the prefabricated facilities can be deployed using normal transport, e.g., rail, truck, boat, cargo plane or helicopter, to a location in need. The container, depending on circumstance, can be equipped with an electrical generator and supplied with a dedicated accompanying fuel source to supply the generator when warranted, for example, in disaster circumstances. However, the preferred embodiment is to be delivered to and actuated to meet surge demand, for example at a postal facility, a parking lot, a stadium, an open field, on the back of a trailer truck, etc., i.e., wherever is advantageous for optimizing throughput.

In the Covid19 pandemic, to date, urine itself has not been shown to contain functioning virions. SARS-CoV-2 RNA assayed in saliva samples is the accepted positive indicator of infection. Less than 10 percent of SARS-CoV-2 positive patients had detectable viral RNA in the urine. So robust genetic tracing of the virus is not possible in urine. Since urine is so commonly available for many bioassays including the presently discussed VOC assays, use of urine as a diagnostic sample and assaying urine VOCs becomes the rational diagnostic/screening method. Fine-tuning signature sensitivity and protocols can be indicative of which patients need more critical care and which may be getting sicker or recovering. A decrease in viral disease signature may be taken as a sign that the host has or has not resolved the infection.

Cancer

The systems of the present invention are ideally suited for detecting, diagnosing and evaluating cancers. Cancers do not involve all the cells in the body but originate within a specific cell population. As the metabolic activity in the cancer cells is altered, their VOC production changes. Some metabolic changes are common to many cancers. So in some embodiments a signature may be taken as indication of a class of cancers, e.g., epithelial cell cancer. But as signature information is refined the evidence appears to show that even different breast cancers can be distinguished. It is well known that some cancers' responses to hormones if different from other cancers.

Autoimmune Disease

There is evidence to support that many autoimmune diseases are suitable target for analysis using systems of this invention. In general, a signature immune response underlies the autoimmune cascade. When the attacked cells respond, their metabolisms are particular to the attacked cell type, cell age, location and the like. The specialized metabolisms of the altered cells will produce their particular signature VOC outputs. Thus, VOC analysis in accordance with this invention will be expected to assist in diagnosing various autoimmune diseases.

Microbiome

Another exemplary application is understanding of a person's microbiome. Surface microbes generally emit only trace amounts of VOCs and thus are not yet prime targets for analysis. But ambient gases surrounding an organism may be analyzed to obtain signature information emitted from the skin, whether from the organism or its associated microbiome. In a related set of analyses, since the gut microbiome directly feeds into the bloodstream for filtration by the kidneys this information can be more concentrated and provide a stronger signal. VOCs produced by the organism and the gut microbiome will therefore be present in general analysis. Thus, this microbiome status can be monitored in accordance with the present invention.

Sample Alternatives to Urine

This brings up an alternative application of the present invention. While initial development focused on urine analysis, urine being available non-invasively and in decent volumes; persons also are used to providing sterile urine samples so sample collection is not a confounding issue, the inventor has become aware that inventive technologies are broadly applicable with only minor adjustments. Off-gassing of stool samples can be directly measured. Solid tissue biopsies can be stored and allowed to off-gas into a head space for analysis. The solid sample may be disrupted mechanically and/or liquefied to provide a liquid sample closer to those samples originally conceived. Blood, plasma, lymph, saliva and mucous, though not as readily available as urine are easily obtained samples that are suitable VOC sources for introduction into the device of this invention.

Specialized Applications

Low or Zero Gravity

The device of the invention is engineered for specific applications. For example, in a weightless or low gravity environment, the random movements of molecules in the vapors will offer significant advantages over devices that assay liquid samples. Collection, storage and feeding of samples into the device will consider the effect or non-effect of gravity, but within the assay chamber and on the chip gravitational effects on the vapor and molecular attraction will be negligible.

The devices and methods of the present invention may serve as a component part of a larger device or larger method. The device is not required to operate in isolation. Data obtained using assays of the present invention may be intercalated with other data, e.g., medical history, age, residence, etc., during alalyses. Additional information may be obtained using the sample being assayed in the primary device of the invention. For example, in an enhanced machine, a capillary analysis system, in series or less preferably in parallel, might be used as one variable or parameter in the data processing to guide analysis along a preferred branch or if used in parallel confirm or question results from the VOC and, when so configured, large molecule analysis structures of the present invention.

In another embodiment, a FET or similar sensor system as known in the art to assay components in a liquid phase may be associated with the vapor phase analysis of the present invention in an accessory or complementary device. The large molecule sensor chips may reside on a separate chip or in a separate compartment than VOC analytical components. Large biomolecules such as proteins are not found in VOC off-gas. Assays of antibody and many antigenic larger molecules can add to the assay information obtained using the base system of the present invention. Such information, especially IgM or IgG status can help delineate a patient's historical experience with a disease. Such information can also be helpful in determining the efficacy of immunizations and/or the frequency of recommended booster immunizations. SWNT-based biosensor diagnostic devices in contact with an analyte containing liquid have emerged the current millennium as effective high sensitivity detectors for medical, industrial, environmental, toxicological, quality control, pharmaceutical development, etc., applications. Neutral and ionic compounds in aqueous solutions including, but not limited to: insulin, human chorionic gonadotropin, human growth hormone, prolactin, glucose, fructose, galactose, hormones, neurotransmitters, drugs, amino acids, peptides, proteins, products of micro-organisms — including pathogens and microbiome members, cancer indicative nucleic acids and proteins, etc. have been investigated using such technologies. In a recent review article, electrical, optical, electrochemical, outputs were characterized as sensed signals. Szunerits, S., & Boukherroub, R. (2018). Graphene-based biosensors. Interface focus, 8(3), 20160132. https://doi.org/10.1098/rsfs.2016.0132. Optical properties include light transmission (transparency), light changing (fluorescence), reflecting, and absorbing properties of graphene in various formats. Antigen-antibody complexes are detectable.

Such add-on device could be advantageous when the presence of a large (non-volatile) molecule might be important information. Accordingly, an embodiment of the present invention may incorporate a liquid phase detection component to augment data obtained in the vapor phase.

As an illustrative example of a multi-analysis device, would be one in which two or more types of chips are used. The first type of chip is that of the base device described above to produce the VOC signature. The second type is a chip that analyzes a wet or liquid phase sample. While many alternative form factors may be used, a cartridge (e.g., containing a liquid to off gas the vapors for assay by the first chip) can be configured to incorporate a second type chip that includes sensors for e.g., viral nucleic acid, antigen and or antibodies or other non-volatile compounds of interest. Other configurations may optionally deliver samples to such a liquid phase analysis chip in parallel to delivering vapors to the gas phase sensors. Thus, data from multi-analysis procedures on the same sample can be collated and analyzed together.

Another illustrative example embodies a relatively large protein attached to or in close proximity to the liquid-based sensor element(s). Such protein may be a protein with affinity for the molecule in question. When binding said molecule and thus changing its 3-D structure a signal can result in detection of the molecule in question. Such sensor component might be selected to provide immune status, e.g., presence of one or more classes of antibodies or target of the antibody. Enzymes, hormones, hormone receptors, neuro-active compounds and receptors are examples of large molecules or proteins that might be assayed in such liquid phase analysis. In some embodiments of the invention the liquid phase and vapor phase may be assaying different components of the same event, such as an antibody active in lung that might bind SARS-CoV-2 or the liquid phase sensor might sense a VOC that can be assayed in the vapor phase thereby helping to increase confidence in the results. This dual phase multi-analytical system is an advance over existing systems as it provides, from a single sample, a detailed understanding of an individual's health status.

1. A nanosensor device sensitive to volatile organic compounds, said device comprising a sensing element comprising:
a first sensor layer;
a second sensor layer;
a base support;
an electrical input; and
a signal output;
said first and second layers each comprising graphene surfaced nanosensing elements wherein at least said second sensor layer comprises single walled carbon nanotubes (SWNTs);
said nanosensing elements decorated or functionalized with a bioattractive compound;
at least said second layer configured to assay compound in a gas phase.

2. The nanosensor device of claim 1 wherein said first sensor layer comprises non-tubular graphene (ntG), said first sensor layer disposed to accept and assay compounds present in a liquid.

3. The nanosensor device of claim 2 wherein said second sensor layer assays volatile organic compounds (VOCs) and said first sensor layer assays non-volatilized organic compounds (nVOCs).

4. The nanosensor device of claim 3 wherein said nVOCs comprised molecules having a molecular weight about 400 g/mol or greater.

5. The nanosensor device of claim 3 wherein said VOCs comprised molecules having a molecular weight about 400 g/mol or less.

6. The nanosensor device of claim 1 wherein said bioattractive compound comprises a nucleic acid.

7. The nanosensor device of claim 6 wherein said nucleic acid comprises a plurality of bases selected from the group consisting of: adenine (A), cytosine (C), guanine (G), thymine (T), and uracil (U).

8. The nanosensor device of claim 1, wherein said first sensor layer comprises SWNTs, said second sensor layer having a diameter approximately equal to or less than a diameter of said first layer, said first and second sensor layers stacked upon the same base support and wherein said first and second sensor layers detect gas phase VOCs and modify an electronic signal of their base support.

9. The nanosensor device of claim 8 further comprising a third sensor layer, said third sensor layer spaced from said first layer by at least said second layer, said second layer having a diameter greater than a diameter of said third layer.

10. The nanosensor device of claim 1 further comprising a heat source.

11. The nanosensor device of claim 10 wherein said heat source augments volatilizing compounds from a liquid phase.

12. The nanosensor device of claim 10 wherein said heat source heats said base support.

13. The nanosensor device of claim 1 comprising a plurality of sensing elements.

14. The nanosensor device of claim 13 comprising at least about 2⁸ sensing elements.

15. The nanosensor device of claim 13 comprising at least about 2¹⁰ sensing elements.

16. The nanosensor device of claim 13 wherein said plurality of sensing elements are disposed on a columnar surface.

17. The nanosensor device of claim 16 wherein said plurality of sensing elements are disposed on an inner surface of a tube or column.

18. The nanosensor device of claim 2 wherein said first sensor layer comprises crumpled graphene.

19. The nanosensor device of claim 1 further comprising at least one inert layer.

20. The nanosensor device of claim 19 wherein said at least one inert layer comprises graphene.

26. The nanosensor device of claim 20 wherein at least one inert layer is disposed in a zone between said sensors that preferentially interact with compounds whose molecular weight median value is greater than about 400 g/mol and said support supporting at least said first and second layers.

27. The nanosensor device of claim 1 wherein said second sensor layer is on a strand continuous with said first layer, said second layer being disposed by wrapping in a spiraling pattern atop said first layer.

28. The nanosensor device of claim 1 wherein first and second sensor layers comprise SWNTs and wherein a third layer comprising ntG is disposed more proximal to said base support than said first layer.

29. The nanosensor device of claim 2 wherein said first and second layers are disposed on a solid or hollow rod or shaft, said device further comprising an elevator controlled to expose sample to the sensing layer comprising ntG while a SWNT sensor layer remains surrounded by gas.

30. The nanosensor device of claim 29 further comprising a heating element configured to heat sample liquid and augment vaporization of VOCs.

31. The nanosensor device of claim 29 comprising a support for a solid sample, and wherein said elevator brings said solid sample in contact with said sensing layer comprising ntG.

32. The nanosensor device of claim 29 wherein said first layer is disposed on a wall of said rod or shaft in a location more proximal to the sample contact point than said second layer.

33. The nanosensor device of claim 32 wherein said first sensor layer is disposed on the inner or the outer wall of said rod or shaft and said second sensor layer is disposed on the outer or the inner wall of said rod or shaft, respectively.

34. The nanosensor device of claim 29 wherein said elevator comprises separate controls for the sensing layer comprising ntG and at least one said SWNT sensor layer.

35. A method for fabricating a nanosensor device of claim 1, said method comprising:
disposing a first SWNT layer on said support at a site on said support having an electrical terminal;
decorating said first SWNT layer with a bioattractive compound;
disposing a second SWNT layer atop said decorated first SWNT layer in an amount resulting in a diameter of said second SWNT layer being less than the diameter of said first SWNT layer; and
decorating said second SWNT layer with a bioattractive compound.
